(19) United States
(12) Patent Application Publication
(10) Pub. No.: US 2012/0039959 A1
(43) Pub. Date: Feb. 16, 2012

(54) Anti-Adhesion Alginate Barrier of Variable Absorbance

(76) Inventors: Joerg TESSMAR, Regensburg (DE); Eva Esser, Regensburg (DE); Thomas Reintjes, Regensburg (DE); Lukas Bluecher, Eurasburg (DE); Michael T. Milbocker, Holliston, MA (US)

(21) Appl. No.: 13/211,226

(22) Filed: Aug. 16, 2011

Related U.S. Application Data
(60) Provisional application No. 61/374,218, filed on Aug. 16, 2010.

(51) Int. Cl. A61K 9/00; A61P 43/00; A61K 47/36

(52) U.S. Cl. 424/400; 514/779

(57) ABSTRACT

Described are mono- and bi-layer alginate post-surgical anti-adhesion barriers with tailored absorption profiles and non-migrating characteristics. Muco-adhesive properties of alginates in their solid state are used to localize the device, and lubricious properties of alginates in their liquid state are used to mitigate adhesion formation during wound healing. In addition, the design of the implant can be selected such that the crosslinking agent is released from the device under specific conditions and the absorbance profile modified. A medicinal agent may optionally be incorporated.

### CROSS-REFERENCE TO RELATED APPLICATIONS

This application claims the benefit of U.S. Provisional Application No. 61/374,218, filed Aug. 16, 2010 and entitled Alginates for Adhesion Preventing Films (Att. Docket MB8402PR2), which is related to U.S. Provisional Application No. 61/353,157, filed Jun. 9, 2010 and entitled Crosslinked Alginate Film (Att. Docket MB8402PR), the entire contents both of which are expressly incorporated herein by reference. This application is related to U.S. application Ser. No. 12/480,655, filed Jun. 8, 2009 (Att. Docket MB8110P), U.S. application Ser. No. 12/498,291, filed Jul. 6, 2009 (Att. Docket MB8134P), U.S. application Ser. No. 10/660,461, filed Sep. 10, 2003 (Att. Docket MA9758P), now U.S. Pat. No. 7,704,520, U.S. application Ser. No. 10/019,797, filed Jul. 26, 2002 (Att. Docket MB9962P), U.S. application Ser. No. 10/385,399, filed Mar. 10, 2003 (Att. Docket MA9496CON), now U.S. Pat. No. 6,673,362, U.S. application Ser. No. 10/631,980, filed Jul. 31, 2003 (Att. Docket MB9604P), now U.S. Pat. No. 7,592,017, U.S. application Ser. No. 11/203,660, filed Aug. 12, 2005 (Att. Docket MB9828P) and U.S. application Ser. No. 12/199,760, filed Aug. 27, 2008 (Att. Docket MB8039P), The foregoing applications are commonly assigned, and the entire contents of all of them are expressly incorporated herein by reference.

### BACKGROUND OF THE INVENTION

1. Field of the Invention

The present invention relates generally to medical devices and, more particularly, to devices and methods for preventing the formation of adhesions between a healing trauma site and adjacent surrounding tissue, possessing an adhesive functionality directed toward the healing tissue surface to prevent mobilization of the adhesion barrier after implantation and a post-surgical anti-adhesion functionality that becomes increasingly more rapidly absorbed by a mammalian body after implantation.

2. Description of Related Art

Surgery or injury often leads to the problem of internal tissue adhesions which can cause pain and restrictions in movement. Injury, surgical incision or abrasion to, for example, the peritoneum, pleural or abdominal cavity can result in an outpouring of a serosanguinous exudate. The exudate subsequently coagulates, producing fibrinous bands between abutting surfaces which can become organized by fibroblast proliferation to form collagenous adhesions.

Adhesion formation following surgery often results in chronic pain. For example, adhesions that form in relation to intestinal surgery, e.g., bowel resection, hernia repair, etc. may cause obstruction of the intestine. Adhesions that form within the pelvic area may reduce or hinder the normal movement of the area of repair by restricting the natural relative movement of tissue layers, Adhesions may also form in the vicinity of nerves and disrupt nerve transmissions with a resultant diminution of sensory or motor function.

Approaches to reduction of post-surgical adhesion include the application of drugs or surfactants, and the use of collagen, collagen-fabric, collagen membranes or reconstituted collagen as physical barriers. Other barriers are made from hyaluronic acid, polylactic acid, amino acid polymers and chitin.

In situ methods of barrier formation have utilized carboxyl-containing polysaccharides. Barriers can consist of a polysaccharide solution, covalently cross-linked polysaccharide or ionically cross-linked polysaccharide.

Other materials used to form physical barriers in an attempt to prevent adhesions include silicone elastomers, gelatin films and knit fabrics of oxidized regenerated cellulose. In other instances, anti-coagulants such as heparin, heparinoid, or hexuronyl hexosaminogly are incorporated into a matrix of biocompatible material, such as matrices of hyaluronic acid, cross-linked and uncross-linked collagen webs, synthetic resorbable polymers, gelatin films, absorbable gel films, oxidized cellulose fabrics and films.

In particular, alginate complexes have been used in a variety of applications. U.S. Pat. No. 4,267,240 describes a novel release sheet comprising a web of paper with a water-soluble, alkaline earth or earth metal salt, e.g. a calcium salt such as calcium chloride and then coated on said sized side with a film of a mixture of a salt of alginic acid, and either (1) a triglyceride or (2) hydrolyzed or non-hydrolyzed lecithin.

U.S. Pat. No. 4,505,935 describes a water-soluble alginate and an aqueous dispersion of hydrophilic lipid crystals. A calcium salt is then applied on the surface of the ointment, which converts the alginate to insoluble calcium salt.

U.S. Pat. No. 5,096,754 describes a film having a base layer of a material which may be fiber-reinforced, wherein the material includes a mixture of cellulose hydrate and alginic acid and/or alginate. The alginate may be the calcium salt of alginic acid.

U.S. Pat. No. 5,484,604 describes a transdermal drug delivery device comprising a polymer matrix of sodium alginate and nicotine casted over a backing material and sprayed with a solution of calcium ions to cross-link.

U.S. Pat. No. 5,508,043 describes a controlled release matrix of sodium alginate and a calcium salt.

U.S. Pat. No. 5,596,084 describes a gel comprising water, sodium ions, calcium ions, and about 0.3 and 4% alginate.

U.S. Pat. No. 5,670,169 describes an alginate based hydrating gel system for the purpose of treating wounds that need moisture.

U.S. Pat. No. 5,684,051 describes an elastically deformable medical device of a polymer of polysaccharide-based hydrogel, such as barium alginate,

U.S. Pat. No. 5,981,821 describes a matrix of calcium alginate associated with at least one alginate of a multivalent metal, with the exception of magnesium.

U.S. Pat. No. 6,022,556 describes a wound dressing material comprising an alginate ester of a polyhydric alcohol; a humectant consisting of one or more monohydric or polyhydric alcohols; and water,

U.S. Pat. No. 46,150,581 describes post-surgical anti-adhesion barriers, methods of preventing post-surgical adhesions, and methods and devices for forming post-surgical anti-adhesion barriers containing alginate.

U.S. Pat. No. 6,451,351 describes a gel composition, such as alginate gel beads, using a proper concentration of calcium pantothenate or calcium ascorbate as a gelling agent.

U.S. Pat. No. 6,565,901 describes a gel mix of sodium and/or potassium alginate and a slowly-soluble calcium salt, with the calcium salt being incorporated in a crystalline sugar.

U.S. Pat. No. 6,638,917 describes a method of reducing adhesion at a site of trauma by forming a film from an alginate solution, contacting the film with a cross-linking solution to form a cross-linked mechanically stable sheet, and placing at least a portion of the sheet at the site of trauma.

U.S. Pat. No. 6,693,089 describes a method of reducing adhesion at a site of trauma including forming a film from an alginate solution.

U.S. Pat. No. 7,612,029 describes a substrate comprising a nonwoven layer containing an ionically crosslinked alginate polymer used to control the release of active ingredients.

U.S. Pat. No. 7,879,362 describes a prolonged/controlled release of a medicinal preparation containing alginate.

### SUMMARY OF THE INVENTION

The invention generally involves low cost, easy to place and reposition anti-adhesion barrier sheets. Prior methods and devices for reduction of trauma site adhesion have several deficiencies. For example, some of these deficiencies are post-implantation migration, dissolution prior to wound healing, fractionation of implant resulting in focal fibrotic centers, and localization of fluid.

The invention also generally involves adhesion barriers that have low cost and are easy to use. Adhesion barriers according to the invention do not require in situ formation, have a lifetime in a body of up to two weeks or more, and permit a medical worker to both reposition and fix the barrier at a desired location. The invention generally relates to a repositionable, long life, low cost barrier sheet that a medical worker need not suture to tissue. The invention also generally relates to a drug delivery device.

In one aspect, the invention features a device for insertion into a body to block adhesion between layers of healthy tissue and a layer of compromised tissue. The device comprises a sheet comprising ionically cross-linked alginate, the crosslinker of which diffuses into the body such that the sheet initially has robust mechanical properties but rapidly degrades after a time, typically two weeks, after which tissue adhesions are not formed. The sheet has sufficient mechanical stability to provide an effective barrier to adhesion formation prior to the time of dissolution.

In one embodiment, the sheet has a thickness in a range of 0.25 mm to 10 mm. In a further embodiment, the sheet has a tear strength in a range of 5 psi to 500 psi. In a further embodiment, the sheet can be fabricated, or cut by a medical worker, in a variety of shapes, including a polygon, an oval and a disk.

In one embodiment, an inner portion of the sheet or side against the compromised tissue layer has a lower density of cross-linking relative to an outer portion of the sheet or side against healthy tissue, such that the inner side is more slippery and the outer side is mucoadhesive. In one aspect, the invention features a method of forming a sheet for use as an adhesion barrier. The method comprises forming a film from an alginate solution, and contacting the film with a cross-linking solution to form a cross-linked mechanically stable sheet.

In another embodiment, the alginate solution comprises an additive for medical treatment, for example, an antiseptic, an antibiotic, an anticoagulant, a contraceptive, a nucleic acid molecule, a protein, and generally a drug.

In another embodiment, the alginate solution comprises a biocompatible dye to assist observation of sheet location in a body. In other embodiments of the invention, a filler or other additive is included in the cross-linking solution.

This invention relates to controllably absorbable polymeric medical devices for insertion into a body and methods for making such devices. More particularly, the invention relates to cross-linked alginate barriers for reduction of post-surgical body tissue adhesion which are self adhesive and prevent migration of the implant. The medical devices according to the invention are suitable for both human and animal use.

Alginates are hydrophilic marine biopolymers with the unique ability to form heat-stable gels that can develop and set at physiologically relevant temperatures. Alginates are a family of non-branched binary copolymers of 1-4 glycosidically linked .beta.-D-mannuronic acid (M) and .alpha.-L-guluronic acid (G) residues. The relative amount of the two uronic acid monomers and their sequential arrangement along the polymer chain vary widely, depending on the origin of the alginate.

The relative content of G and M monomers in the alginate polymers affects pore size, stability and biodegradability, gel strength and elasticity of gels. Alginate polymers contains large variations in the total content of M and G, and the relative content of sequence structures also varies largely (G-blocks, M-blocks and MG alternating sequences) as well as the length of the sequences along the polymer chain. Generally, the lower the G content relative to M content in the alginate polymers used the more biodegradable a gel will be. Gels with high G content alginate generally have larger pore sizes and stronger gel strength relative to gels with high M alginate, which have smaller pore sizes and lower gel strength.

Mechanical properties of the present implants can also be modified by the addition of crosslinkers to the alginate either in the pre-cured liquid state or after casting into sheets in the solid state. Whereas, utilizing the innate structure of alginates to design desired absorbance profiles is useful, the use of crosslinkers provide an additional versatility wherein the crosslinker can be designed to elute from the alginate substrate, thus temporally reducing the crosslink density of the implant. In addition, by utilizing the solubility of certain salt crosslinkers, one can design an implant of the present invention where the crosslinker is released into the implant after the implant is placed into a mammalian body by the action of hydration. Finally, a surgical site may be treated with a crosslinker to modify an implant of the present invention to augment either the anti-adhesive property of the implant on a preferred side or alternatively the adhesivity of the implant.

The invention has application in various surgical procedures, such as: 1) gynecological in which procedures of myomectomy via laparotomy or laparoscopy where during removal of a fibroid, an incision is made in the uterus, and a barrier can be placed in between the uterus and the surrounding tissues to prevent adhesion; 2) abdominal procedure where an adhesion barrier can be used to prevent peritoneal adhesions and therefore prevent intestinal obstruction; 3) cardiac procedure where a barrier can be used to prevent post-operative adhesion after cardiac procedures which require removal of the pericardium; 4) cranial procedure where a barrier can protect the exposed cortex during craniotomy to prevent the skull and the cortex from adhering; and 5) musculoskeletal procedure where a barrier can prevent adherence of a tendon and the surrounding tissues.

In another aspect, a post-surgical anti-adhesion barrier delivery device includes a first layer designed to dissolve rapidly within a mammalian body and a second layer designed to dissolve slowly within a mammalian body, whereby said first layer becomes slippery within the body before surgical closure of the repair site and said second layer is mucoadhesive and creates an anti-adhesive property between said implant and surrounding tissue. In a method of surgical repair, the slippery side is oriented away from the compromised tissue to shield healthy tissue from a repair site and the adhesive side is oriented toward the repair site to localize the implant to this locus of healing.

While the apparatus and method has or will be described for the sake of grammatical fluidity with functional explanations, it is to be expressly understood that the claims, unless indicated otherwise, are not to be construed as limited in any way by the construction of “means” or “steps” limitations, but are to be accorded the full scope of the meaning and equivalents of the definition provided by the claims under the judicial doctrine of equivalents.

Any feature or combination of features described or referenced herein are included within the scope of the present invention provided that the features included in any such combination are not mutually inconsistent as will be apparent from the context, this specification, and the knowledge of one skilled in the art. In addition, any feature or combination of features described or referenced may be specifically included, replicated and/or excluded, in any combination, in/from any embodiment of the present invention. For purposes of summarizing the present invention, certain aspects, advantages and novel features of the present invention are described or referenced. Of course, it is to be understood that not necessarily all such aspects, advantages or features will be embodied in any particular implementation of the present invention. Additional advantages and aspects of the present invention are apparent in the following detailed description and claims that follow.

### BRIEF DESCRIPTION OF THE DRAWINGS

The invention will be described by reference to the following drawings, in which like numerals refer to like elements, and in which:

FIG. 1a illustrates the absorbance profile for an exemplary implant of the resent invention when placed in a liquid medium containing 0 mg of calcium.

FIG. 1b illustrates the absorbance profile for an exemplary implant of the resent invention when placed in a liquid medium containing 0.6 mg of calcium.

FIG. 1c illustrates the absorbance profile for an exemplary implant of the resent invention when placed in a liquid medium containing 1.2 mg of calcium.

FIG. 2a illustrates load and strain at maximum load for an exemplary implant of the present invention when the implant is modified with glycerol.

FIG. 2b illustrates strain at break for an exemplary implant of the present invention when the implant is modified with glycerol.

FIG. 3a illustrates load and strain at maximum load for an exemplary implant of the present invention when the implant is modified with PEG.

FIG. 3b illustrates strain at break for an exemplary implant of the present invention when the implant is modified with PEG.

### DETAILED DESCRIPTION OF THE INVENTION

Embodiments of the invention are now described and illustrated in the accompanying drawings, instances of which are to be interpreted to be to scale in some implementations while in other implementations, for each instance, not. In certain aspects, use of like or the same reference designators in the drawings and description refers to the same, similar or analogous components and/or elements, while according to other implementations the same use should not. According to certain implementations described or referenced herein, use of directional terms, such as, top, bottom, left, right, up, down, over, above, below, beneath, rear, and front, are to be construed literally, while in other implementations the same use should not. The present invention may be practiced in conjunction with various implant fabrication and other use techniques that are conventionally used in the art, and only so much of the commonly practiced process steps and features are included herein as are necessary to provide an understanding of the present invention. The present invention has applicability in the field of medical devices and processes in general. For illustrative purposes, however, the following description pertains to a thin sheet implant and related methods of manufacture.

Post-surgical anti-adhesion barriers, methods of preventing post-surgical adhesions, and methods and devices for forming post-surgical anti-adhesion barriers are provided. The adhesion barriers can be mono-layer or bi-layer, depending on the application. When biological liquid of the wound comes into contact with the anti-adhesion barrier the metal ions of one, or one of two layers of alginates, are released, without dissolution. Before the ions of the other alginate are also released to the point of hydroelectrolytic equilibrium between the ions of the biological liquid and tissue, the ions released restore the physiology of the cells of the wound by provision of ions of the multivalent metal. Thus the dormant metabolism of the cells at the base of the wound can be reawakened.

Controlled absorbance of alginate anti-adhesion barriers as described herein prevent formation of post-surgical adhesions at a wound or trauma site by interposing a unique biocompatable, bioabsorbable barrier between damaged tissue and adjacent surrounding tissue.

As described in more detail below, the anti-adhesion barrier may contain calcium as a crosslinking agent, but other multivalent metals of the alginate associated with the calcium alginate matrix is advantageously selected from the group comprising zinc, manganese, copper, selenium, barium.

Whether a single composition of alginate is employed wherein the formed layer is first strongly crosslinked and thus adhesive, but there after by diffusion of the crosslinker out of the layer becomes slippery, preferably within a time required for exudate proteins to denature and localize the implant, or whether a two layer device is employed wherein one layer has diminished crosslinking, the functionality of the crosslinker is central.

Appropriate cross-linking cations include, but are not limited to, alkaline earth metals, such as calcium, magnesium, barium, strontium, and beryllium ions; transition metals, such as iron, manganese, copper, cobalt, zinc, and silver ions; other metallic elements, such as boron, aluminum, lead, and bismuth ions; and polyammonium ions.

Alternatively, calcium scavenging anions or chelating compounds can be employed, suitable anions are derived from polybasic organic or inorganic acids. Appropriate cross-linking anions include, but not limited to, phosphate, sulfate, citrate, borate, succinate, maleate, adipate and oxalate ions. Or alternatively hardly soluble EDTA (Ethylenediaminetetraacetic acid) salts can be added, which later complex released calcium.

Preferred cross-linking cations are calcium, iron, and barium ions. The most preferred cross-linking cations are calcium and barium ions. The most preferred cross-linking anion is phosphate. Cross-linking may be carried out by contacting the polymers with an aqueous solution containing dissolved ions.

The relative concentration of crosslinker to alginate determines the crosslink density. The higher the crosslink density, the slower the dissolution of the implant in situ.

Alternatively, absorbance profile and mechanical properties of the implants of the present invention can be modified by the addition of excipients to alginate films as they cure. Suitable excipients are generally alcohols, and include glycerol, propylene glycol and polyethylene glycol, which are differently effective, but can be used to adjust the film properties. Alternative approaches of modification include the chemical conjugation of the alginate with the softeners in order to obtain soft polymer films at the implantation site.

Other plasticizers, generally by groups, are phthalates, trimellitates, adipates, sebacates, maleates, benzoates, epoxidized vegetable oils, sulfonamides, and organophosphates.

### Modification of Crosslink Density

With respect to the goals of the present invention, suitable embodiments are those constructs which transition from an adhesive state to a slippery anti-adhesive state as a function of time, or alternatively, the implant is spatially differentiated, and manufactured with two sides of differing crosslink density.

Generally, the cross link density can be modified during manufacture of implants of the present invention by three different methods. One method embodiment comprises the incubation of the alginate film with a solution of crosslinker, for example calcium lactate, via spraying or alternatively via dipping or rinsing. Another method embodiment comprises “inner gelation” initiated during the casting of an alginate film containing a hardly soluble salt (e.g., calcium salt) and gluconolacton, which decreases the pH upon hydrolysis and dissolves the metal salt initiating the crosslinking process. Yet another method embodiment comprises application of a hardly soluble metal salt (e.g., calcium salt), which is finally dissolved via spraying the prepared films with lactic acid solution. All different procedures aim to adjust the concentration of crosslinker (e.g., calcium) within the final medical implant, which ultimately defines the device absorbance profile and elimination from the patient.

In creating a two-sided functionality in the implant, the goal of the present invention is to possess macroscopically a single layer implant in which one side possesses a higher crosslink density. One approach is to crosslink a sheet of alginate at a relatively high level, and then reduce the crosslink density on one side of the implant.

In one embodiment, displacement of cross-linking ions from one side of the sheet can be accomplished by applying a solution containing a stripping agent to one side of the sheet. The stripping agent serves to displace, sequester, or bind, the cross-linking ions present in the ionically cross-linked polymer, thereby removing the ionic cross-links. Some stripping agents are polyions capable of forming stable ionic bonds with the cations or anions disclosed above.

The choice of any particular stripping agent will depend on whether the ion to be displaced is an anion or a cation. If the cross-linking agent is a cation, then the stripping agent will be a polyanion, while if the cross-linking agent is an anion, the stripping agent will be a polycation. Suitable stripping agents include, but are not limited to, organic acids and their salts or esters, phosphoric acid and salts or esters thereof sulfate salts and alkali metal or ammonium salts.

Examples of stripping agents include, but are not limited to, ethylene diamine tetraacetic acid, ethylene diamine tetraacetate, citric acid and its salts, organic phosphates, such as cellulose phosphate, inorganic phosphates, such as, pentasodium tripolyphosphate, mono and dibasic potassium phosphate, sodium pyrophosphate, phosphoric acid, trisodium carboxymethyloxysuccinate, nitrilotriacetic acid, maleic acid, oxalate, polyacrylic acid, as well as sodium, potassium, lithium, calcium and magnesium ions.

In other embodiments, the stripping step or alternatively the crosslinking step is accomplished by dipping or spraying the sheet on one side. Some electrolytes for stripping are chlorides of monovalent cations such as sodium, potassium or lithium chloride, as well as other stripping salts described above. The solution may also contain plasticizing ingredients, such as glycerol or sorbitol, to facilitate inter- and intra-polymer chain motion for shaping of the sheet or deriving desired mechanical characteristics of the sheet.

Other approaches to varying the properties of an alginate sheet include varying the composition of alginate itself. Alginates are the salt and ester forms of alginic acid. Alginate is a polymer made up of guluronic acid and mannuronic acid. By varying the amount of guluronic acid and mannuronic acid present in alginate, physical properties such as gel strength and film forming properties are varied. Stronger less dissolving films result from a higher relative concentration of guluronic acid.

Naturally occurring alginates with known varying concentrations of guluronic acid and mannuronic acid are commercially available. The molecular weight of the alginates used herein may range from about 200,000 to several million depending on the source of the alginate. Alginate is a polyanionic polymer having functionalized carboxyl groups. Preferred alginate salts for use herein are sodium and potassium salts. Methods of dissolving alginate in water are well-known by those with skill in the art. As above, distilled water, sterile water and bacteriostic water are suitable for use herein. The second solution may also be made isotonic.

Variations, modifications, and other implementations of what is described herein will occur to those of ordinary skill in the art without departing from the spirit and the scope of the invention as claimed. Accordingly, the invention is to be defined not by the preceding illustrative description but instead by the spirit and scope of the claims.

All the chemical components listed in these examples can be purchased from Sigma-Aldrich, USA, unless otherwise indicated.

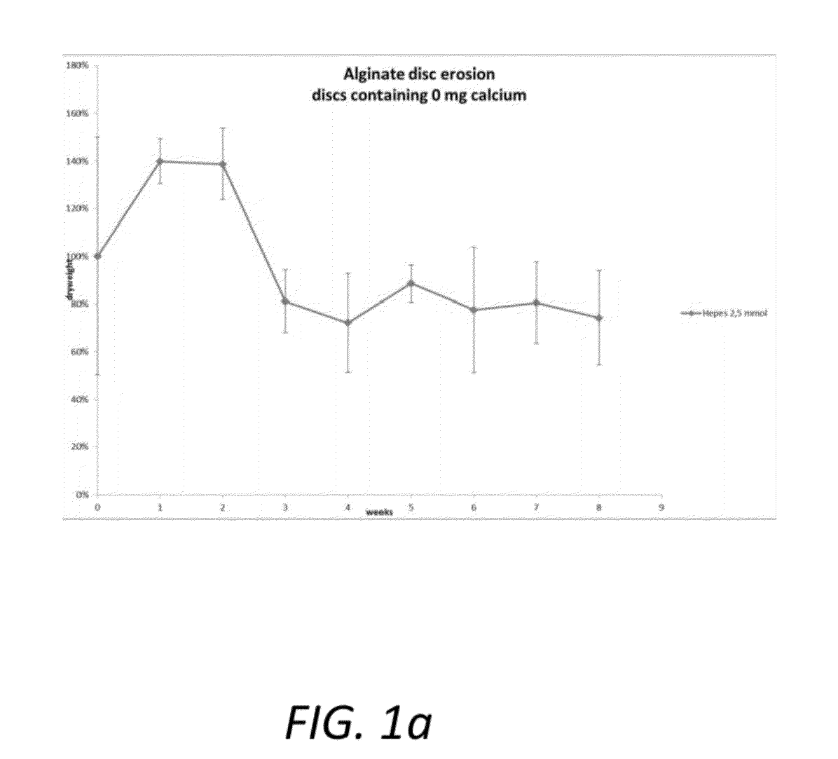

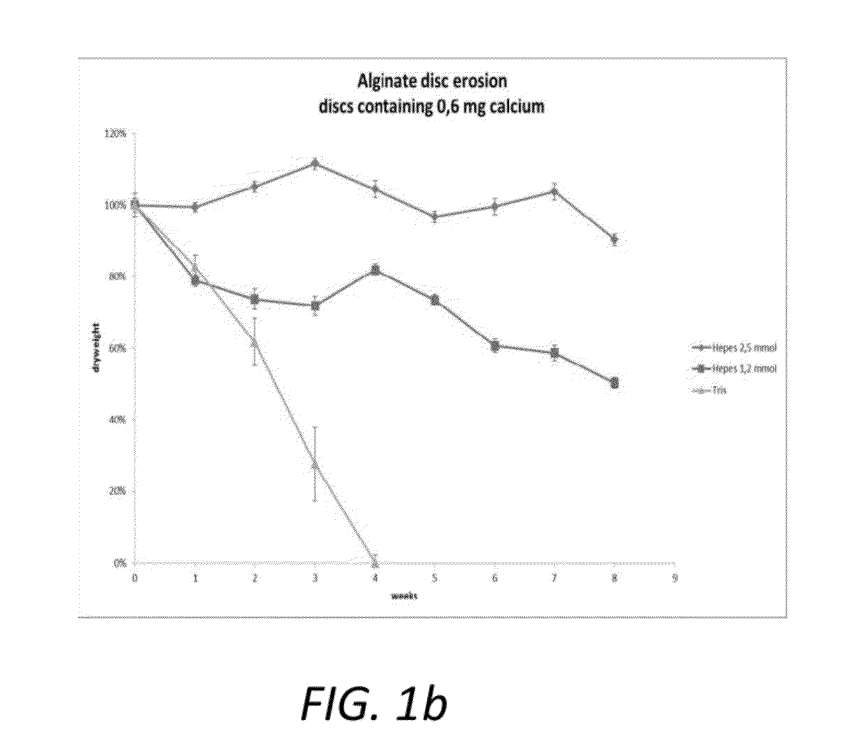

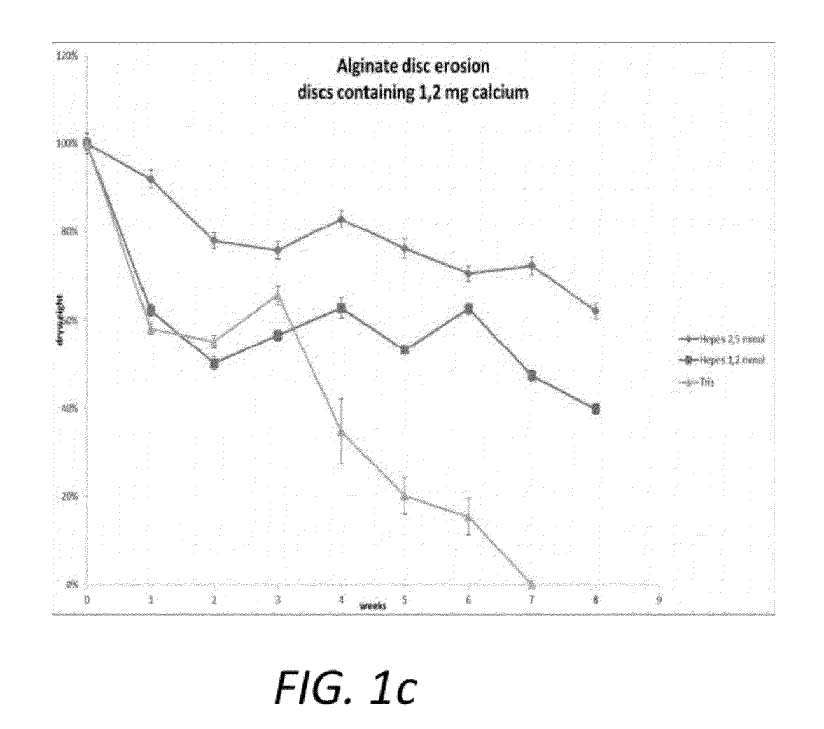

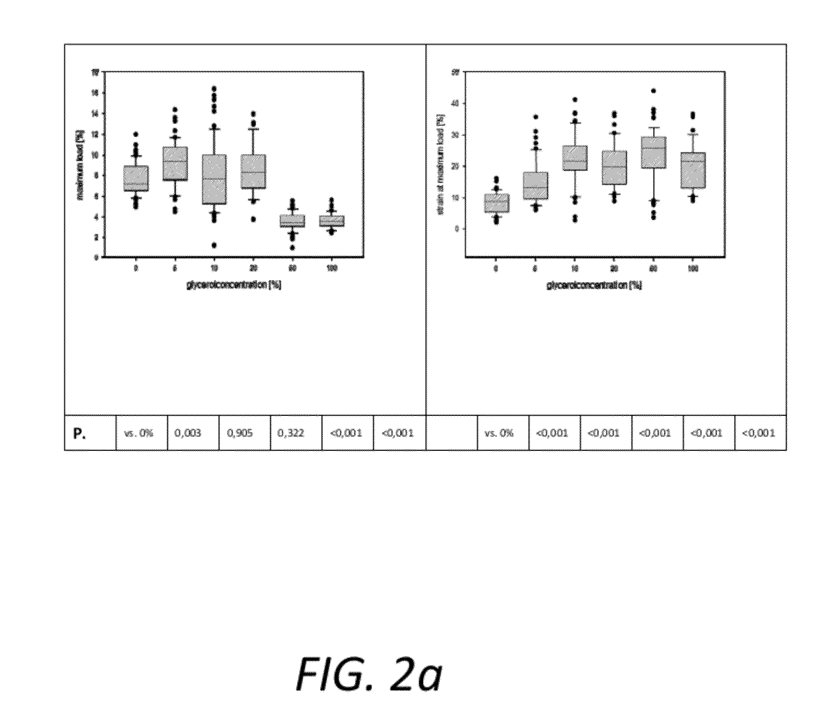

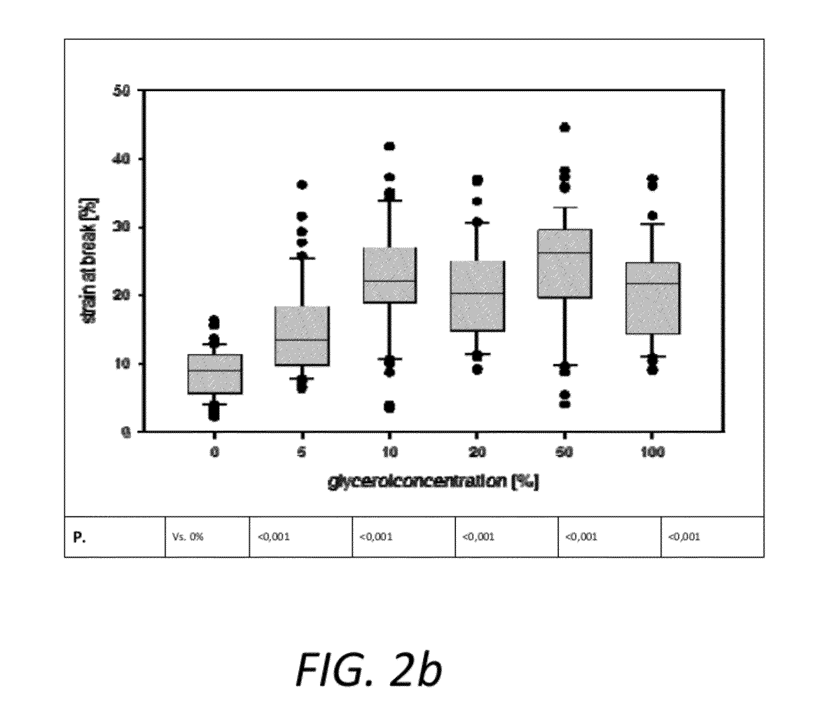

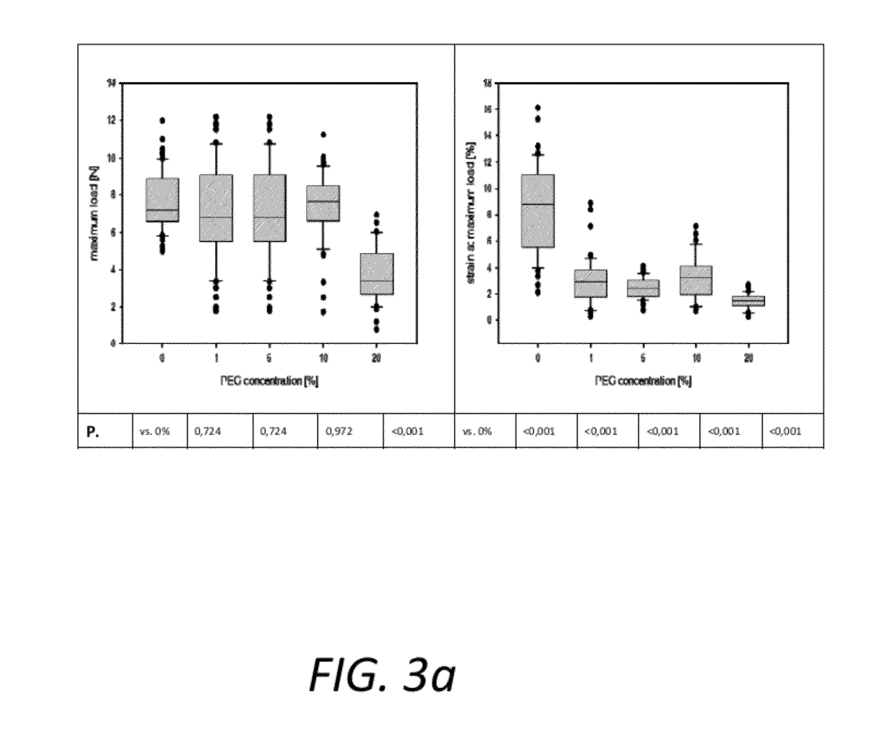

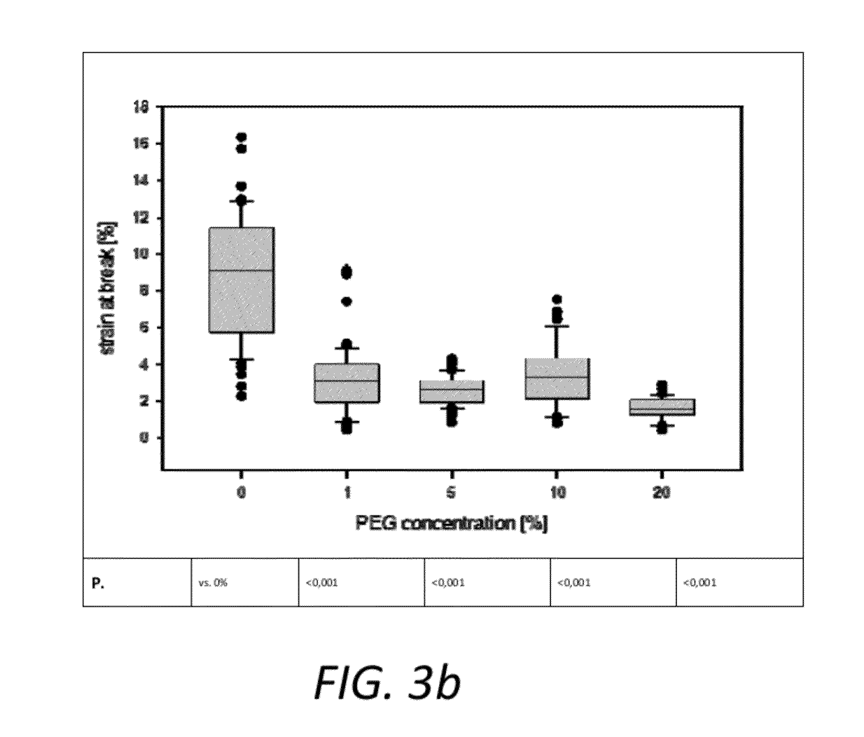

Referring more particularly to the drawings, FIGS. 1a,b,c, illustrate variation in absorbance profile (weight loss vs time) for an exemplary implant of the present invention when placed in three different liquid media of varying calcium content; FIGS. 2a,b illustrate variation in mechanical strength profile for an exemplary implant of the present invention when the implant is modified with glycerol; and FIGS. 3a,b illustrate variation in mechanical strength profile for an exemplary implant of the present invention when the implant is modified with polyethylene glycol (PEG).

### Example 1a

### Alginate Mucoadhesive Sheet

6 g of alginate LF 10/60 and 6 g of Glycerol are dissolved in 100 g of Millipore water. This alginate solution is cast on a glass slide with an ERICHSEN coatmaster 509 MC, with a gap clearance of 700 μm. The emerging film is subsequently sprinkled, using a vaporizer, with a calcium lactate solution containing 4% calcium lactate in Millipore water. After 5-10 minutes reaction time, the procedure is repeated several times, until 20 ml of the calcium lactate solution has been sprinkled over the film. After drying about 72 h, the film can be peeled off the glass slide.

### Example 1b

### Alginate Mucoadhesive/Anti-Adhesive Sheet

6 g of alginate LF 10/60 and 6 g of Glycerol are dissolved in 100 g of Millipore water. This alginate solution is cast on a glass slide with an ERICHSEN coatmaster 509 MC, with a gap clearance of 700 μm. The emerging film is subsequently sprinkled, using a vaporizer, with a calcium lactate solution containing 2% calcium lactate in Millipore water. After 5-10 minutes reaction time, the procedure is repeated several times, until 20 ml of the calcium lactate solution has been sprinkled over the film. After drying about 72 h, the film can be peeled off the glass slide.

### Example 2

### Alginate Anti-adhesive Sheet

6 g of alginate LF 10/60 and 6 g of Glycerol are dissolved in 100 g of Millipore water. This alginate solution is cast on a glass slide with an ERICHSEN coatmaster 509 MC, with a gap clearance of 700 μm. The emerging film is subsequently sprinkled, using a vaporizer, with a calcium lactate solution containing 2% calcium lactate in Millipore water. After 5-10 minutes reaction time, the procedure is repeated several times, until 5 ml of the calcium lactate solution has been sprinkled over the film. After drying about 72 h, the film can be peeled off the glass slide.

### Example 3

### Alginate Anti-Adhesion Sheet with Localizing Side

A first layer is constructed by combining 6 g of alginate LF 10/60 and 6 g of Glycerol dissolved in 100 g of Millipore water. This alginate solution is cast on a glass slide with an ERICHSEN coatmaster 509 MC, with a gap clearance of 700 μm. The emerging film is subsequently sprinkled, using a vaporizer, with a calcium lactate solution containing 4% calcium lactate in Millipore water. After 5-10 minutes reaction time, the procedure is repeated several times, until 20 ml of the calcium lactate solution has been sprinkled over the film. After drying about 72 h, the film can be peeled off the glass slide.

These films are cut into circles in a manner such they closely fit into a Petri dish. On top of this layer is poured an alginate solution as prepared above. As the film and the newly added solution begin to solidify, the surface is sprinkled with a calcium lactate solution containing 2% calcium lactate in Millipore water. The sprinkling is continued until 5 ml of calcium lactate solution is sprinkled over the film. After drying, the two-sided film can be pealed from the Petri dish. The resulting construct possess a higher density of calcium on one side than the other side. The higher content calcium side is the mucoadhesive side and the lower calcium content side is the anti-adhesive side.

### Example 4

### Method of Implantation

A sheet constructed according to Example 2 is implanted in a mammalian body. The sheet is adhesive and can be place, pealed up, and replaced until a final desired location is achieved. Then a solution of physiologic saline containing 2% calcium lactate is sprayed on the surface of the implant proximal to the tissue defect surface. The calcium crosslinks the proximal surface of the implant, making it more mucoadhesive. Alternatively, a sheet constructed according to Example 1a is implanted in a mammalian body. A stripping solution is applied to the distal side, to reduce the calcium content on the distal surface and increase its anti-adhesive function.

### Example 5

### Test Articles for Degradation Study

A degradation study was conducted. Test articles were alginate discs fabricated from alginate LF 10/60, with 2 cm diameter, containing (mg, 0.6 mg and 1.2 mg calcium. These discs were produced via the method of “inner gelation” described below.

The first compound of the inner gelation method consists of 144 trig alginate and 144 mg glycerol dissolved in 14.4 ml Millipore water. The second compound is a suspension containing 72 mg or 144 mg calcium citrate, 288 trig gluconolactone and 5.8 ml Millipore water.

After the Millipore water is given to the components of the suspension, the resulting solution is vortexed for 15 seconds and given to the alginate solution. This mixture is also vortexed for 15 seconds. Within 2 minutes this mixture is poured into a Teflon dish of 72 cm2. Over time the gluconolactone decreases the pH slowly. With this pH decrease the calcium dissolves from its citrate complex and the crosslinking of the alginate takes place.

After a gelation time of approximately 10 hours the still wet alginate film can be cut into discs.

### Example 6

### Test Articles for Mechanical Testing

For the mechanical testing alginate films containing different amounts of the plasticizers glycerol and polyethylene glycol [PEG] were prepared.

For the film preparation either 3 g of alginate LF 10/60 or 2 g of alginate LF 10/60 FT and different amounts of glycerol or PEG are dissolved in 50 g of Millipore water. Calcium citrate, a hardly soluble calcium salt is added to the solution with the help of an Ultraturrax mixer. Films were cast with an ERICHSEN coatmaster 509 MC (gap clearance=700 μm), to obtain thin films, which were then sprinkled with lactic acid to dissolve the calcium and initiate cross-linking. The films were dehydrated at 23±2° C. and 50±5% relative humidity.

For mechanical testing, the films were cut with a razor blade into uniform strips (1×5 cm or 1×17.5 cm).

Test Article Preparation Matrix

| plastiziser concentration | addition according to | addition according to |
|---|---|---|
| refering to the alginate | 2 g LF 10/60 FT | 3 g LF 10/60 |
| 0% | 0 g | 0 g |
| 1% | 0.02 g | 0.03 g |
| 5% | 0.1 g | 0.15 g |
| 10% | 0.2 g | 0.3 g |
| 20% | 0.4 g | 0.6 g |
| 50% | 1 g | 1.5 g |
| 100% | 2 g | 3 g |

### Example 7

### Degradation of Test Articles Made According to Example 4

Degradation was accomplished by placing test articles in buffer solutions formulated as described below:

HEPES buffer 1.2 mmol/l Ca2+ pH 7.4 (adjusted with Na(H)

### 0.26 g HEPES

### 0.818 g NaCl

### 4.8 mg CaCl2

ad 100 ml Millipore water
HEPES buffer 2.5 mmol/l Ca 2+ pH 7.4 (adjusted with NaOH)

### 0.26 g HEPES

### 0.818 g NaCl

### 10.1 mg CaCl2

ad 100 ml Millipore water
Tris buffer pH 7.4 (adjusted with HCl):

### 0.61 g Tris

### 3.7 ml HCl 1N

ad 100 ml Millipore water

During the degradation study, buffer solutions were exchanged weekly.

### Results:

Test articles placed in Tris-buffer were eroded completely after 4 to 5 weeks (FIG. 1a). The discs with the lower content of calcium eroded faster than the discs with the higher content. Alginate discs without calcium. Test articles were completely dissolved in Tris buffer in HEPES buffer containing 0.6 mg (1.2 mmol/l Ca 2+) calcium (FIG. 1b). The discs in HEPES buffer containing 1.2 mg calcium (2.5 mmol/l Ca2+) (FIG. 1c) incorporated the calcium from the buffer. As a result, there occurred additional crosslinking after starting the degradation study (those points>100%). Therefore these discs initially became heavier than the initial mass.

### Example 8

### Mechanical Testing of Films According to Example 5

Mechanical testing was conducted on alginate films containing different amounts of the plasticizers glycerol and polyethylene glycol [PEG]. The films were prepared according to Example 3.

The mechanical testing was carried out with a texture analyzer (Instron 5542). The films were tested according to an American national standard “Standard Test method for Tensile Properties of Thin Plastic Sheeting D 882-02”. The texture analyzer is of the constant rate-of-crosshead-movement type. It has a stationary member carrying one grip, where one end of the test specimen was fixed and a movable member carrying a second grip, were the other end of the specimen was fixed. The load under strain was measured by a load cell with a capacity of 50 N.

The environmental conditions were 23±2° C. and 50±5% relative humidity. The 5 cm strips were fixed within the grips with 3 cm of the strip bridging the grips. The 17.5 cm long strips were fixed with 12.5 cm of the strip between the grips.

Crosshead speed was 10 nm/min. When a minimum load of 0.5N was reached during the testing of the 5 cm stripes, the crosshead speed was increased to 100 mm/min. The 17.5 cm stripes were tested with a speed of 12.5 mm/min after a minimum load of 0.1 N was reached, Test articles were elongated until it ruptured. Rupture was defined at the point where the load suddenly decreases by about 40%. The maximum load [%], strain at maximum load [%] and strain at break [%] were recorded and calculated by the INSTRON® software Bluehill®2 version 2.16.

### Results:

Increasing amounts of glycerol positively affected the maximum load of the glycerol containing films (FIG. 2a). The strain at break was also changed by the used glycerol concentration (FIG. 2b). Higher concentrations than 10% glycerol didn't show a greater effect on the tensile properties.

These effects were less pronounced with PEG, which could eventually be covalently attached to the alginate (FIGS. 3a and 3b).

The addition of plasticizers also has a significant impact on the mechanical properties of the alginate based adhesion barriers.

### Alternative Methods and Compositions

Molecular weight, crosslink density, porosity, ratio and structure of M- and G-blocks of the implants of the present invention affect the absorbance profile as well as the mucoadhesive and anti-adhesive properties.

The mucoadhesivity can be further enhanced by the addition of disulfide bridges. When thiol groups are added to the alginate casting solution and oxidized by air the mechanical properties of the resulting films are strengthened. For example, thiol groups can be used to increase the stability of the films. Alternatively, when the disulfide bridges are not utilized internally then the thiol groups are available for binding to SH groups located in living tissue. For example, alginate may be modified with cysteine to obtain an implant of the present invention.

Alginate cross-linking is done mainly by incorporation of calcium ions, which link neighboring acid groups. One possible application would be the addition of calcium complexing phosphate, citrate or EDTA which removes calcium from the film and leads to a spontaneous dissolution. Other cross-linking schemes have to include chemical links which can be added to the film and break upon a chemical or enzymatical trigger.

Corresponding or related structure and methods disclosed or referenced herein and/or in any and all co-pending, abandoned or patented application(s) by any of the named inventor(s) or assignee(s) of this application and invention, are incorporated herein by reference in their entireties, wherein such incorporation includes corresponding or related structure (and modifications thereof) which may be, in whole or in part, (i) operable and/or constructed with, (ii) modified by one skilled in the art to be operable and/or constructed with, and/or (iii) implemented/made/used with or in combination with, any part(s) of the present invention according to this disclosure, that of the application and references cited therein, and the knowledge and judgment of one skilled in the art.

Although the disclosure herein refers to certain illustrated embodiments, it is to be understood that these embodiments have been presented by way of example rather than limitation, Corresponding or related structure and methods specifically contemplated, disclosed and claimed herein as part of this invention, to the extent not mutually inconsistent as will be apparent from the context, this specification, and the knowledge of one skilled in the art, including, modifications thereto, which may be, in whole or in part, (i) operable and/or constructed with, (ii) modified by one skilled in the art to be operable and/or constructed with, and/or (iii) implemented/made/used with or in combination with, any parts of the present invention according to this disclosure, include: (I) any one or more parts of the above disclosed or referenced structure and methods and/or (II) subject matter of any one or more of the following claims and parts thereof, in any permutation and/or combination, include the subject matter of any one or more of the following claims, in any permutation. The intent accompanying this disclosure is to have such embodiments construed in conjunction with the knowledge of one skilled in the art to cover all modifications, variations, combinations, permutations, omissions, substitutions, alternatives, and equivalents of the embodiments, to the extent not mutually exclusive, as may fall within the spirit and scope of the invention as limited only by the appended claims.

What is claimed is:

1. A device for preventing post-surgical adhesions, comprising:
one or more compositions of alginate;
a crosslinking agent; and
combining alginate and crosslinking agent to form a solid anti-adhesion barrier to treat a site of surgical intervention.

2. A device according to claim 1, wherein said alginate composition contains a plasticizer.

3. A device according to claim 2, wherein said plasticizer is a polyol.

4. A device according to claim 2, wherein said plasticizer is selected from a list comprising phthalates, trimellitates, adipates, sebacates, maleates, benzoates, epoxidized vegetable oils, sulfonamides, and organophosphates.

5. A device according to claim 1, wherein two alginate compositions are employed, one with a lesser amount of crosslinking agent and the second containing a greater amount of crosslinking agent, wherein a first layer is formed of the first alginate composition and in juxtaposition a second layer is formed of the second alginate composition such that when implanted into a mammal one layer becomes slippery and the other layer is adherent to living tissue.

6. A device of claim 1, wherein said crosslinking agent is applied to said alginate composition after said alginate is in sheet form.

7. A device according to claim 1, wherein the crosslinking agent is selected from the group consisting of calcium chloride, calcium citrate, calcium sulfate, magnesium chloride, magnesium citrate and magnesium sulfate.

8. A device of claim 1, wherein said crosslinking agent is released from said device into the body of a mammal subsequent to implantation, such that the rate of absorption of said alginate composition in the mammalian body is faster than if the amount of crosslinking agent had stayed constant subsequent to implantation within said device.

9. A device according to claim 1, wherein said crosslinking agent solution and said alginate sheet are combined by spraying said solution onto a target site within a mammalian body at the site of surgical intervention prior to implantation of said sheet such that said crosslinking agent and said alginate sheet combine in situ.

10. A device according to claim 1, wherein a medicinal agent is added.

11. A device according to claim 1, wherein thiol groups are added to add cleavable crosslinks.

12. A device according to claim 1, wherein calcium groups are employed which are slowly solubilizing.

13. A device according to claim 1, wherein chelating substances are employed to cause rapid loss of cations.

14. A device of claim 13, wherein said chelating substance is a calcium scavenging substance.

15. A device of claim 14, wherein said calcium scavenging substance is EDTA.

16. A device according to claim 1, wherein said anti-adhesion barrier is formed in situ.

17. A device according to claim 1, wherein said device promotes living cellular healing and neovascularization of a tissue defect and discourages acellular fibrosis.

18. A method utilizing the device of claim 1, wherein placement of the device of claim 1 in a tissue defect promotes healing while minimizing scar tissue formation.
